(19) United States
(12) Patent Application Publication
(10) Pub. No.: US 2012/0022330 A1
(43) Pub. Date: Jan. 26, 2012

(54) Tactile indicator for endoscopic instruments

(76) Inventor: Robert H. Meichner, Fairhope, AL (US)

(21) Appl. No.: 12/805,324

(22) Filed: Jul. 26, 2010

(51) Int. Cl. A61B 1/00

(52) U.S. Cl. 600/121

(57) ABSTRACT

A flexible endoscopic instrument for insertion through the lumen of an endoscope, comprises: a flexible cable; an actuating handle connected to a proximal end of the flexible cable; a tool assembly connected to a distal end of the flexible cable; a sheath that covers substantially an entire length of the flexible cable; and a tactile indicator portion formed along an outer surface of the sheath so as to be tactilely detectable by a user. The tactile indicator portion is formed at a predetermined distance from the distal end of the flexible cable so as to be tactilely detectable by the user at a position before the tactile indicator portion is inserted into a proximal end of the lumen of the endoscope and before the tool assembly exits a distal end of the lumen. When the flexible cable of the instrument is inserted into the endoscope, the user is pushing the instrument into the lumen of the endoscope, whereby he/she is physically touching the surface of the flexible cable's sheath. The user is thus able to tactilely sense progress of the flexible cable as it enters the lumen. When the user feels the presence of the tactile indicator portion on the sheath, this indicates that the distal end of the instrument has nearly emerged out of the distal end of the endoscope's lumen. By observing the tactile indicators, the user can then determine how much further the instrument can be inserted in order to emerge out of the endoscope without damaging nearby tissue.

### BACKGROUND OF THE INVENTION

1. Field of the Invention

The invention relates to flexible microsurgical instruments. More particularly, the invention relates to a flexible microsurgical instrument which has a tactile and a visual position indicator.

2. State of the Art

Flexible microsurgical instruments, in particular endoscopic biopsy forceps, are used for introducing a variety of tools, instruments and medicaments into the human body without having to resort to extremely invasive surgical procedures. These instruments are typically designed as long flexible coils containing one or more control wires coupled to actuating handles at a proximal end of the flexible instrument. The actuating handle moves the control wires relative to the cable to effect the desired operation at the distal end of the coil.

For example, in endoscopic biopsy forceps, a pair of forceps jaws are mounted on a clevis at the distal end of the coil. The forceps jaws are coupled to the control wires so that movement of the control wires causes the jaws to open and close to bite a tissue sample.

An endoscopic biopsy procedure is accomplished through an endoscope which is inserted into a patient's body and manipulated to a biopsy site. The endoscope typically includes a long narrow flexible tube with an optical lens and a lumen for receiving the biopsy forceps or other flexible instruments. Practitioners, such as gastroenterologists, guide the endoscope to the biopsy site while looking through the optical lens and insert the biopsy forceps through the lumen of the endoscope to the biopsy site. While viewing the biopsy site through the optical lens of the endoscope, the practitioner manipulates the actuating handle to effect a tissue sampling operation at the distal end of the instrument. When first introducing the instrument through the endoscope, the practitioner must carefully insert the instrument through the endoscope while holding the actuating handle to maintain the jaws in a closed position. Otherwise, the jaws, which are typically spring-mounted, may open suddenly when the jaws exit out of the distal end of the endoscope in the patient's body. In addition, if the practitioner introduces the instrument through the endoscope too quickly or with too much force, or if the practitioner does not carefully monitor the progress of the instrument's cable through the endoscope, the distal end of the instrument may exit the end of the endoscope's lumen with too much force and impact the surrounding tissue. In either situation, the distal end of the flexible instrument will cause internal injuries to the patient.

Even worse, such injuries may not have any immediate effect but rather have delayed or long term impact. For example, the perforation of a hollow viscous that is being biopsied may go undetected during the procedure. If such injury is either ignored or unnoticed at the time, delays in the treatment of the injury will have fatal consequences.

Even more, the causing of such injury may give rise to claims of medical malpractice against the practitioner or the institution in which the practitioner performed the procedure. If the practitioner failed to follow the proper protocols or standard of care for performing the procedure so as to minimize the potential of causing any injury, such failure would substantially impact the practitioner's or the institution's liability.

Consequently, there exists a need for some mechanism by which a practitioner using an endoscope in conjunction with a flexible instrument can monitor the progress of the flexible instrument through the endoscope lumen and thereby avoid unnecessarily injuring a patient with the distal end of an instrument as it clears the opening of the endoscope's lumen.

### SUMMARY OF THE INVENTION

It is therefore an object of the invention to provide a means for indicating the position of a flexible instrument inside an endoscope.

It is also an object of the invention to provide both visual and tactile indication of the position of the flexible instrument inside an endoscope.

It is another object of the invention to provide means for indicating the position of an instrument's distal end inside an endoscope which does not interfere with the operation of the instrument or the endoscope.

It is still another object of the invention to provide means for indicating the position of a flexible instrument inside an endoscope which is intuitively easy to use.

It is also an object of the invention to provide means for indicating the position of a flexible instrument inside an endoscope which is inexpensive to manufacture.

Still further, it is an object of the invention to provide a mechanism by which a practitioner or an institution can form a procedure or protocol in the performing of a procedure using flexible instruments inside an endoscope. Such protocols or procedures would be designed to avoid or at least minimize the potential of causing injury to patients, in order to meet required standards of care.

In accord with these objects which will be discussed in detail below, the present invention includes a flexible sheath which covers a substantial portion of the flexible cable of a flexible microsurgical instrument. The sheath is provided with a series of ribbed tactile indicators formed and positioned at a predetermined distance from the distal end of the microsurgical instrument, depending on the type of endoscope and purpose for which the endoscope and the corresponding instrument are being used. When the flexible cable of the microsurgical instrument is inserted into the endoscope, the practitioner is pushing the instrument into the lumen of the endoscope, whereby he/she is physically touching the surface of the flexible cable's sheath. The practitioner is thus able to tactilely sense progress of the flexible cable as it enters the lumen. When the practitioner feels the presence of the ribbed tactile indicators on the sheath, this indicates that the distal end of the flexible instrument has nearly emerged out of the distal end of the endoscope's lumen. By observing the ribbed tactile indicators, the practitioner can then determine how much further the flexible instrument can be inserted in order to finally emerge out of the endoscope without damaging nearby tissue.

Preferred aspects of the invention include: covering the flexible cable with the sheath either along its entire length or only where the ribbed tactile indicators are formed, fabricating the sheath from TEFLON, high-density polyethylene (HDPE), or other applicable polymeric biomaterials with the ribbed tactile indicators formed thereon so as to be tactilely and visually discernible by a user, and locating the indicators along the length of the sheath depending on the type of instrument and purpose for which the instrument is being used. The ribbed tactile indicators may be formed on or under the sheath using any conventional forming process known in the art, including but not limited to hot stamping the sheath so that it forms the ribbed tactile indicators while also acquiring the texture of the sheath. Additional objects and advantages of the invention will become apparent to those skilled in the art upon reference to the detailed description taken in conjunction with the provided figures.

### BRIEF DESCRIPTION OF THE DRAWINGS

Hereinbelow is a brief description of each of the drawings illustrating the various aspects and embodiments of the present invention, wherein:

FIG. 1 is an overall perspective view of the first embodiment of the present invention during use;

FIGS. 2A and 2B are perspective and side elevation views, respectively, of the first embodiment of the present invention, wherein the shape, position and location of the ribbed tactile indicators are shown;

FIG. 3 is side elevation view of a second embodiment of the present invention;

FIG. 4 is side elevation view of a third embodiment of the present invention; and

FIG. 5 is side elevation view of a fourth embodiment of the present invention.

### DETAILED DESCRIPTION OF THE PREFERRED EMBODIMENTS

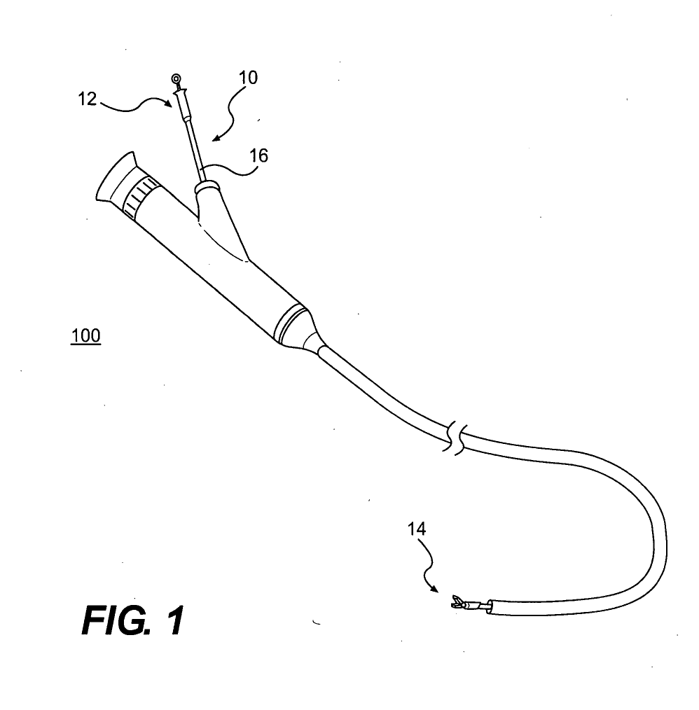

Referring now to the drawings illustrating the various features and embodiments of the present invention, wherein like elements and features are similarly numbered throughout the drawings, FIG. 1 in particular shows a known flexible endoscopic instrument 100 suitable for use with the instrument 10 of the present invention (i.e., biopsy forceps) that generally includes an actuating handle 12 connected to a proximal end of a long flexible cable 16 and a tool assembly 14 connected to a distal end of the cable 16.

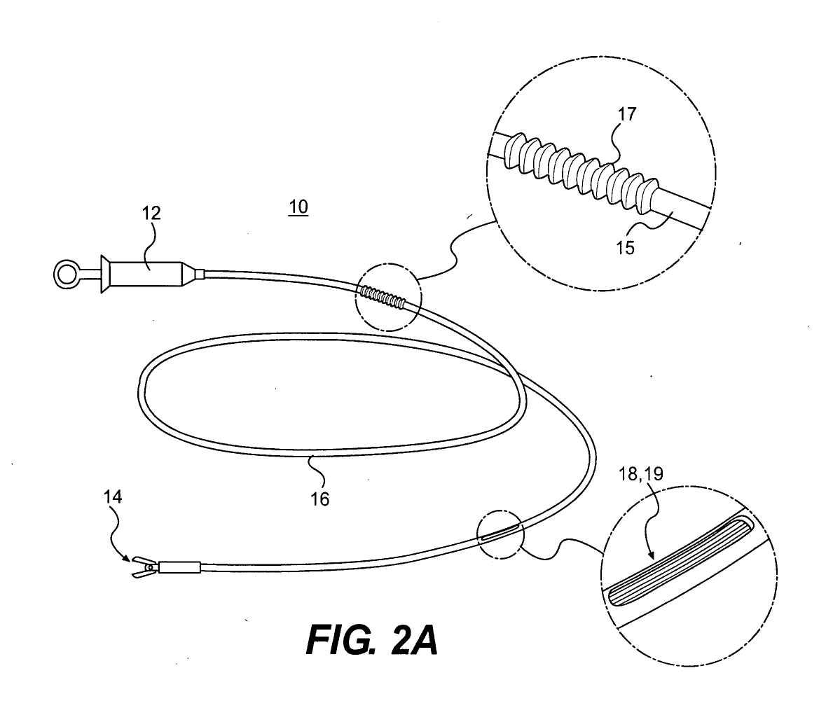

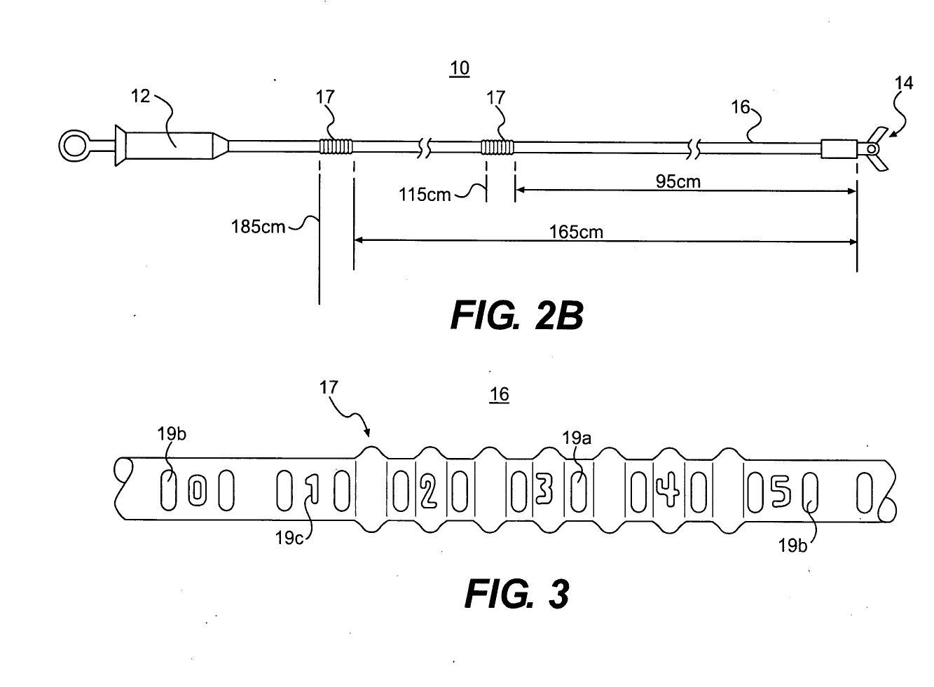

With respect to FIG. 2A, the flexible cable 16 includes a pair of axially displaceable control wires 18,19 extending through the interior length of the cable 16 couples the handle 12 with the tool assembly 14. The cable 16 includes a sheath 15 that covers substantially the entire length of the cable 16. The proximal end of the cable 16 connects to the actuating handle 12 and the distal end of the cable 16 is connected to the tool assembly 14, in this case the biopsy forceps. The sheath 15 of the cable 16 is formed from TEFLON, high-density polyethylene (HDPE), or other applicable polymeric biomaterials using any conventional forming process known in the art, including but not limited to hot stamping the sheath so that it forms tactile indicators 17 while also acquiring the texture of the underlying cable 16. The tactile indicators 17 are specifically formed thereon so as to be tactilely and visually discernible by a user; in other words, a user can both see the tactile indicators 17 along the length of the sheath 15, and feel the tactile indicators 17 between his/her fingers as he/she pushes the cable 16 into the lumen of an endoscope. In at least this first embodiment of the present invention, the tactile indicators 17 are formed as a series of ribbed tactile indicators. The detection of the ribbed tactile indicators 17 by the user is an immediate indication that the distal end of the instrument 10 is on the verge of exiting the endoscope's lumen and impacting into the surrounding tissue of the patient's internal organs.

Depending on the type of instrument and purpose for which the instrument is being used, the ribbed tactile indicators 17 may be formed at specific positions or locations along the length of the sheath 15. For example, referring to the first embodiment's implementation of biopsy forceps, as shown in FIG. 2B, if the endoscopic device with which the biopsy forceps are to be used is a 100 cm gastroscope, then one implementation of the invention would have the ribbed tactile indicators 17 formed between 95 cm to 115 cm from the distal end of the instrument 10. Similarly, if the endoscopic device with which the biopsy forceps are to be used is a 170 cm colonoscope, then one implementation of the invention would have the ribbed tactile indicators 17 formed between 165 cm to 185 cm from the distal end of the instrument 10.

In either embodiment, as shown in FIG. 3, the ribbed tactile indicators are formed along a 20 cm portion of the sheath 15. In at least a second embodiment of the invention, the ribbed tactile indicators 17 can also be formed with graduations 19a,19b such that, as the instrument 10 is fed incrementally more into the endoscopic device, the graduations can give a user observing the ribbed tactile indicators 17 at least an approximate determination of how far or how deep the distal end of the instrument 10 (i.e., the biopsy forceps) has entered into the body of the patient. In this embodiment, graduations 19a can be formed as part of the ribbed tactile indicators 17, or graduations 19b can be formed along the length of the sheath 15 either in front of or behind the portion of the sheath 15 on which the ribbed tactile indicators 17 are positioned. The graduations can further be formed to include alphanumeric characters 19c to further aid in indicating the relative position of the instrument 10 within the sheath 15.

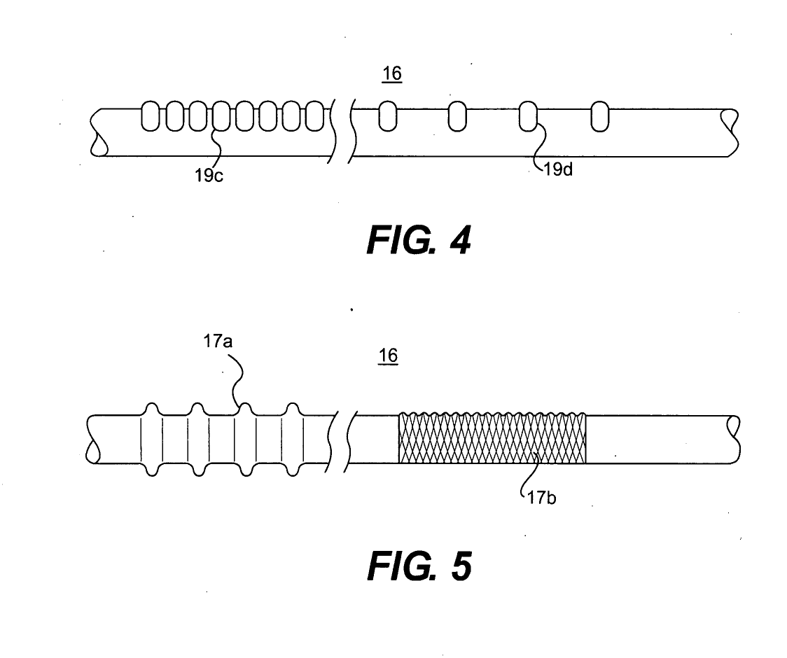

Thus, when a user is conducting a procedure using the instrument 10 in conjunction with an endoscopic device, the user tactilely feels for the ribbed tactile indicators 17 in order to warn when the distal end of the instrument is about to enter, and then the user can use the graduations 19a,19b to visually monitor the extent to which the distal end of the instrument 10 has entered. Alternatively, the graduations 19c can themselves be formed on the sheath as ribbed tactile indicators themselves on the sheath 15 (see FIG. 4).

As a further embodiment, as shown in FIG. 4, the graduations 19c can be formed with different increments than those of other graduations 19d formed on the sheath 15. For example, graduations 19c which are formed closer to the proximal end of the instrument 10 or closer to the ribbed tactile indicators 17 can be formed with smaller increments, while graduations 19d which are formed closer to the distal end of the instrument 10 or further away from the ribbed tactile indicators 17 are formed with larger increments. With this embodiment, when a user is conducting a procedure using the instrument 10 in conjunction with an endoscopic device, the user can first visually or tactilely monitor the graduations 19d to determine the extent to which the instrument 10 is being inserted into the endoscopic device. When the user then observes or feels the graduations 19c with the smaller increments, the user can then start feeling for the ribbed tactile indicators 17 in order to even more accurately warn when the distal end of the instrument 10 is about to enter. Finally, the user can use the graduations 19c to more carefully monitor the extent to which the distal end of the instrument 10 has actually entered.

The graduations may be formed as simple hash marks or alphanumerics along the sheath 15. As noted above, the graduations can themselves be formed on the sheath as ribbed tactile indicators themselves on the sheath 15, as shown in FIG. 4. In addition, the graduations or even the ribbed tactile indicators 17 can be formed in different colors to contrast with the color of the sheath 15, thereby being more visible or noticeable to the human eye, or to indicate positions/locations along the length of the instrument 10. For example, graduations which are formed closer to the proximal end of the instrument 10 or closer to the ribbed tactile indicators 17 can be formed in brighter or fluorescent colors (i.e., yellow, orange, white), while graduations which are formed closer to the distal end of the instrument 10 or further away from the ribbed tactile indicators 17 are formed with less bright colors (i.e., red, blue, green).

In an even further embodiment, as shown in FIG. 5, the tactile indicators 17a can be formed with a different texture than that of tactile indicators 17b formed on another portion of the sheath 15. This may be done in combination with the use of different colors as noted above. In a manner similar to that used with the different increments of graduations, indicators 17a which are formed closer to the proximal end of the instrument 10 can be formed with a more pronounced or noticeable texture, while indicators 17b which are formed closer to the distal end of the instrument 10 or further away from the proximal end are formed with a less pronounced or . less noticeable texture. With this embodiment, when a user is conducting a procedure using the instrument 10 in conjunction with an endoscopic device, the user can first visually or tactilely monitor the indicators 17b to determine the extent to which the instrument 10 is being inserted into the endoscopic device. When the user then observes or feels the indicators 17a, the user is then more accurately warned that the distal end of the instrument 10 is about to enter. Finally, the user can use the indicators 17a, or in conjunction with graduations, to more carefully monitor the extent to which the distal end of the instrument 10 has actually entered.

As would be understood by those of skill in the art, the construction and implementation of an instrument 10 according to the features and advantages of the present invention can be accomplished using manufacturing techniques well known in the relevant art. In particular, in a flexible endoscopic instrument 10 suitable for use with the present invention, the actuating handle 12 may be formed from any heat resistant plastic used for medical applications, as would be known to those of skill in the art. The long flexible cable 16 and the tool assembly 14 connected to a distal end of the cable 16 may be formed from stainless steel. The cable 16 may further be coated in polytetrafluoroethylene (PTFE) materials like TEFLON, high-density polyethylene (HDPE), or other applicable polymeric biomaterials to form the sheath 15. Additionally, any new materials that may be later developed but which could serve at least the same purpose as those mentioned above, and as would be understood by those of skill in the art to be applicable and/or equivalent to being applied to the present invention, could be substituted for the various components of the invention without deviating from the spirit and scope of the invention.

Although the present invention has been fully described in connection with the preferred embodiment thereof with reference to the accompanying drawings, it is to be noted that various changes and modifications will be apparent to those skilled in the art. Such changes and modifications are to be understood as included within the scope of the present invention as defined by the appended claims, unless they depart therefrom.

I claim:

1. A flexible endoscopic instrument for insertion through the lumen of an endoscope, comprising:
a flexible cable having a proximal end and a distal end;
an actuating handle connected to the proximal end of the flexible cable;
a tool assembly connected to the distal end of the flexible cable;
a sheath that covers substantially an entire length of the flexible cable; and
a tactile indicator portion formed along an outer surface of the sheath so as to be tactilely detectable by a user, wherein the tactile indicator portion is formed at a predetermined distance from the distal end of the flexible cable so as to be tactilely detectable by the user at a position before the tactile indicator portion is inserted into a proximal end of the lumen of the endoscope and before the tool assembly exits a distal end of the lumen.

2. A flexible endoscopic instrument according to claim 1, wherein the tactile indicator portion is formed as a plurality of ribbed tactile indicators formed along a predetermined length of the sheath.

3. A flexible endoscopic instrument according to claim 1, wherein the tactile indicator portion is formed as a plurality of textured tactile indicators formed along a predetermined length of the sheath.

4. A flexible endoscopic instrument according to claim 1, wherein the tactile indicator portion includes a plurality of graduation indices.

5. A flexible endoscopic instrument according to claim 4, wherein the plurality of graduation indices include alphanumeric characters.

6. A flexible endoscopic instrument according to claim 1, further comprising:
at least a first tactile indicator portion and a second tactile indicator portion, wherein the first and second tactile indicator portions are formed at first and second predetermined distances, respectively, from the distal end of the flexible cable so as to be tactilely detectable by the user at positions before at least one of the first and second tactile indicator portions is inserted into a proximal end of the lumen of the endoscope and before the tool assembly exits a distal end of the lumen.

7. A flexible endoscopic instrument according to claim 6, wherein each of the first and second the tactile indicator portion is formed as a plurality of textured tactile indicators, the first tactile indicator portion being formed with a texture that is tactilely different from a texture of the second tactile indicator portion.

8. A flexible endoscopic instrument according to claim 8, wherein at least one of the first and second tactile indicator portions includes a plurality of graduation indices.

9. A flexible endoscopic instrument according to claim 4, wherein at least one of the first and second tactile indicator portions includes alphanumeric characters.
